(19) United States
(12) Patent Application Publication
(10) Pub. No.: US 2022/0044789 A1
(43) Pub. Date: Feb. 10, 2022

(54) NUTRITION FORMULA MANAGEMENT SYSTEM

(71) Applicant: IRYOU JYOUHOU GIJYUTU KENKYUSHO CORPORATION, Fukuoka (JP)

(72) Inventor: Shinkichi HIMENO, Fukuoka (JP)

(73) Assignee: IRYOU JYOUHOU GIJYUTU KENKYUSHO CORPORATION, Fukuoka (JP)

(21) Appl. No.: 17/507,166

(22) Filed: Oct. 21, 2021

Related U.S. Application Data
(63) Continuation-in-part of application No. PCT/JP2020/018592, filed on May 8, 2020.

(30) Foreign Application Priority Data
May 10, 2019 (JP) 2019-089558
Feb. 29, 2020 (JP) 2020-034490

(51) Int. Cl. G16H 20/60

(52) U.S. Cl. CPC G16H 20/60

(57) ABSTRACT

A nutrition formula management system includes symptom/finding term management configured to manage terms for representing symptoms or findings of nutrition formula subjects and nutrition formula subject management configured to represent a list of the symptoms or findings of each of the nutrition formula subjects and a list of formulated nutrients using the terms managed by the symptom/finding term management and the formulated nutrient term management and to record whether the formulated nutrients have been effective against each of the symptoms or findings of each of the nutrition formula subjects.

### FIELD

The present invention relates to a nutrition formula management system that assists a doctor, nutritionist, or the like in formulating appropriate nutrition by providing the doctor or the like with information required to make a determination, for example, as to what diet, drug, or supplement is effective for patients complaining of various symptoms caused by nutritional disorders.

### BACKGROUND

A lack of nutrition is known to cause various disorders to the physical condition. Vitamins are seen as important nutrients that are essential to the extent that humans cannot live without them. Proteins, fats, carbohydrates, minerals, and the like are also essential to maintain life. The amounts of intake of these nutrients and the balance between the amounts of intake also have been known to be important as well.

Also, study for improving the motor and persistence functions of athletes and the like has been advanced recently. Also, it has been discussed that the progression of schizophrenia or cancer can be prevented by administering B vitamins or vitamin C in an amount that is 100 times or more larger than the amount of intake that has been known conventionally.

Also, it has been desired to actively use nutrition intake not only to improve the general nutrition state, but also to overcome nutritional disorders, actively promote health, and prevent or improve diseases. Active use of nutrition intake is believed to require deep knowledge on nutrition science, metabolism science, and the like.

Background art literature relating to the present application includes the following.

### CITATION LIST

### Patent Literature

Patent Literature 1: Japanese Unexamined Patent Application Publication No. 2018-5928
Patent Literature 2: Japanese Unexamined Patent Application Publication No. 2009-178549
Patent Literature 3: Japanese Unexamined Patent Application Publication No. 2001-222635

### SUMMARY

Patients with nutritional disorders have various symptoms, and the nutrition components are not directly measured even in a blood test finding. For this reason, it is often difficult to identify deficient nutrition. There are large differences between individuals, and patients are often simultaneously deficient in multiple nutrients. These make it more difficult to grasp the nutritional condition.

To grasp the nutritional condition, it is necessary to know what metabolic pathway is disordered and what symptom appears for what reason in this case. This requires a deep understanding of nutrition science and metabolism chemistry. However, even the already known metabolic pathways are enormous in amount. To make a nutrition formula for improving the disordered metabolic pathway from the symptom, blood test data, and the like, it is necessary to receive training and obtain clinical experience over a long period of time. For this reason, a nutrition formula that cannot be said to be appropriate or optimum is made in not a few cases.

Also, there has been an attempt to list the complicated relationships between deficient nutrients and symptoms or findings and to suggest a deficient nutrient from the symptoms or findings (Patent Literature 3). However, it is difficult to cover all metabolic pathways. Also, there are also side-effect problems, the influence of the synergy between multiple nutrients, and the like and therefore the symptoms and nutrients do not simply correspond to each other one to one. For these reasons, it is not easy to use this approach.

The present invention has been made to solve the above background art problems, and an object thereof is to provide a nutrition formula assistance system that assists in formulating a nutrient having high effectiveness by accumulating a list of nutrients formulated for nutritional disorders patients having various symptoms or findings and records about whether the nutrients have had effects, side effects, or the like and suggesting nutrients or nutrition pairs that is expected to be effective against the symptoms or findings on the basis of data on the frequencies with which effects, no effect, or side effects have occurred. Another object is to suggest symptoms or findings against which nutrients or nutrition pairs are effective so that the symptoms or findings are referenced when formulating nutrition, to previously grasp a risk that an unexpected side effect may occur, to quickly identify a possible nutrient when a side effect occurs, and to allow for formulating nutrition such that when a side effect occurs, a possible nutrient can be identified quickly and a countermeasure can be taken quickly.

To accomplish the above object, a nutrition formula management system of claim 1 includes (i) symptom/finding term management means configured to manage terms for representing symptoms or findings of nutrition formula subjects, (ii) formulated nutrient term management means configured to manage nutrients to be formulated, as terms, (iii) nutrition formula subject management means configured to represent a list of the symptoms or findings of each of the nutrition formula subjects and a list of formulated nutrients using the terms managed by the symptom/finding term management means and the formulated nutrient term management means and to record whether the formulated nutrients have been effective against each of the symptoms or findings of each of the nutrition formula subjects, (iv) symptom/finding-specific effective nutrient list management means configured to count up, as effective subject frequencies, frequencies with which each of nutrient lists have been effectively formulated against each of the symptoms or findings of each of the nutrition formula subjects, and symptom/finding-specific, effective frequency-based nutrient suggestion means configured to display, with respect to each of symptoms or findings of a new nutrition formula subject, a list of nutrients that are managed by the effective nutrient list management means and that have been effective against the symptoms or findings, along with the effective subject frequencies and to suggest nutrients that seem to be effective against the symptoms or findings of the new nutrition formula subject.

According to the nutrition formula management system of claim 2, the nutrition formula management system of claim 1 includes symptom/finding-specific, ineffective nutrient list management means configured to count up, as ineffective subject frequencies, frequencies with which each of nutrient lists have been ineffectively formulated against each of the symptoms or findings of each of the nutrition formula subjects and symptom/finding-specific, effective-ineffective frequency ratio-based nutrient suggestion means configured to display, with respect to each of symptoms or findings of a new nutrition formula subject, a list of nutrients that have been effective against the symptoms or findings, along with ratios between the effective subject frequencies and the ineffective subject frequencies and to suggest nutrients that seem to be effective against the symptoms or findings of the new nutrition formula subject.

According to the nutrition formula management system of claim 3, the nutrition formula management system of claim 1 or 2 includes symptom/finding-specific effective nutrient pair list management means configured to count up, as effective subject frequencies, frequencies with which nutrient pair lists have been effectively formulated against each of the symptoms or findings of each of the nutrition formula subjects and symptom/finding-specific, effective frequency-based nutrient pair suggestion means configured to display, with respect to each of symptoms or findings of a new nutrition formula subject, nutrient pair lists that have been effective against the symptoms or findings, along with the effective subject frequencies and to suggest nutrient pairs that seem to be effective against the symptoms or findings of the new nutrition formula subject.

According to the nutrition formula management system of claim 4, the nutrition formula management system of any one of claims 1 to 3 includes symptom/finding-specific ineffective nutrient pair list management means configured to count up, as ineffective subject frequencies, frequencies with which nutrient pair lists have been ineffectively formulated against each of the symptoms or findings of each of the nutrition formula subjects and symptom/finding-specific, effective-ineffective frequency ratio-based nutrient pair suggestion means configured to display, with respect to each of symptoms or findings of a new nutrition formula subject, nutrient pair lists that have been effective against the symptoms or findings, along with ratios between the effective subject frequencies and the ineffective subject frequencies and to suggest nutrient pairs that seem to be effective against the symptoms or findings of the new nutrition formula subject.

According to the nutrition formula management system of claim 5, the nutrition formula management system of any one of claims 1 to 4 includes nutrient-specific, symptom/finding-specific effective symptom/finding list management means configured to count up, as effective symptom/finding frequencies, frequencies with which each of nutrient lists have been effective against each of the symptoms or findings of each of the nutrition formula subjects for which each of the nutrient lists have been formulated and nutrient-specific, effective frequency-based symptom/finding suggestion means configured to display, with respect to a nutrient considered to be formulated for a new nutrition formula subject, a list of symptoms and findings that are managed by the nutrient-specific, symptom/finding-specific effective symptom/finding list management means and against which the nutrient has been effective, along with the effective subject frequencies and to suggest appropriateness of formula of the nutrient against a symptom or finding of the new nutrition formula subject.

According to the nutrition formula management system of claim 6, the nutrition formula management system of any one of claims 1 to 5 includes nutrient-specific, symptom/finding-specific ineffective symptom/finding list management means configured to count up, as ineffective symptom/finding frequencies, frequencies with which each of nutrient lists have been ineffective against each of the symptoms or findings of each of the nutrition formula subjects for which each of the nutrient lists have been formulated and nutrient-specific, effective-ineffective frequency ratio-based symptom/finding suggestion means configured to display, with respect to a nutrient considered to be formulated for a new nutrition formula subject, a list of symptoms and findings against which the nutrient has been effective, along with ratios between the effective subject frequencies and the ineffective subject frequencies and to suggest appropriateness of formula of the nutrient against a symptom or finding of the new nutrition formula subject.

According to the nutrition formula management system of claim 7, the nutrition formula management system of any one of claims 1 to 6 includes nutrient-specific, symptom/finding-specific effective symptom/finding list management means configured to count up, as effective symptom/finding frequencies, frequencies with which each of nutrient lists have been effective against each of the symptoms or findings of each of the nutrition formula subjects for which each of the nutrient lists have been formulated and nutrient-specific, effective frequency-based symptom/finding suggestion means configured to display, with respect to a nutrient considered to be formulated for a new nutrition formula subject, a list of symptoms and findings that are managed by the nutrient-specific, symptom/finding-specific effective symptom/finding list management means and against which the nutrient has been effective, along with the effective subject frequencies and to suggest appropriateness of formula of the nutrient against a symptom or finding of the new nutrition formula subject.

According to the nutrition formula management system of claim 8, the nutrition formula management system of any one of claims 1 to 7 includes nutrient pair-specific, symptom/finding-specific ineffective symptom/finding list management means configured to count up, as ineffective symptom/finding frequencies, frequencies with which nutrient pair lists have been ineffective against each of the symptoms or findings of each of the nutrition formula subjects for which the nutrient pair lists have been formulated and nutrient pair-specific, effective-ineffective frequency ratio-based symptom/finding suggestion means configured to display, with respect to a nutrient pair considered to be formulated for a new nutrition formula subject, a list of symptoms and findings against which the nutrient pair has been effective, along with ratios between the effective subject frequencies and the ineffective subject frequencies and to suggest appropriateness of formula of the nutrient pair against a symptom or a finding of the new nutrition formula subject.

According to the nutrition formula management system of claim 9, the nutrition formula management system of any one of claims 1 to 8 includes side-effect symptom/finding management means configured to, when a side effect is caused by a formulated nutrient in the nutrition formula subject management means, record a symptom or a finding of the side effect, nutrient-specific, side-effect symptom/finding list management means configured to count up frequencies with which each of nutrient lists have caused symptoms or findings as side effects to the nutrition formula subjects for which each of the nutrient lists have been formulated, and nutrient-specific, side-effect frequency-based symptom/finding suggestion means configured to, with respect to a nutrition formula subject who has had a side effect, display a list of symptoms or findings that are managed by the nutrient-specific, side-effect symptom/finding list management means and that have been previously observed with respect to the nutrient, along with a nutrition formula subject frequency with which the side effect has occurred and to suggest a nutrient that may have caused the side effect to the nutrition formula subject.

According to the nutrition formula management system of claim 10, the nutrition formula management system of any one of claims 1 to 9 includes side-effect symptom/finding management means configured to, when a side effect is caused by a formulated nutrient in the nutrition formula subject management means, record a symptom or a finding of the side effect, nutrient-specific, side-effect symptom/finding list management means configured to count up frequencies with which nutrient pair lists have caused symptoms or findings as side effects to the nutrition formula subjects for which the nutrient pair lists have been formulated, and nutrient pair-specific, side-effect frequency-based symptom/finding suggestion means configured to, with respect to a nutrition formula subject who has had a side effect, display a list of symptoms or findings that are managed by the nutrient pair-specific, side-effect symptom/finding list management means and that have been previously observed with respect to the nutrient pair, along with a nutrition formula subject frequency with which the side effect has occurred and to suggest a nutrient pair that may have caused the side effect to the nutrition formula subject.

According to the nutrition formula management system 11, the nutrition formula management system of claim 9 or 10 includes side-effect symptom/finding-specific nutrient list management means configured to count up frequencies with which symptoms or findings as side effects in the side-effect symptom/finding management means have been caused by each of nutrient lists formulated for the nutrition formula subjects and side-effect symptom/finding frequency-based nutrient suggestion means configured to, with respect to a nutrition formula subject who has had a side effect, display nutrient lists that are managed by the side-effect symptom/finding-specific nutrient list management means and that have been formulated against a symptom or a finding of the side effect, along with a nutrition formula subject frequency with which the side effect has occurred and to suggest a nutrient that may have caused the side effect to the nutrition formula subject.

According to the nutrition formula management system 12, the nutrition formula management system of claim 9 or 11 includes side-effect symptom/finding-specific nutrient pair list management means configured to count up frequencies with which symptoms or findings as side effects in the side-effect symptom/finding management means have been caused by nutrient pair lists formulated for the nutrition formula subjects and side-effect symptom/finding frequency-based nutrient pair suggestion means configured to, with respect to a nutrition formula subject who has had a side effect, display nutrient pair lists that are managed by the side-effect symptom/finding-specific nutrient pair list management means and that have been formulated against a symptom or a finding of the side effect, along with a nutrition formula subject frequency with which the side effect has occurred and to suggest a nutrient pair that may have caused the side effect to the nutrition formula subject.

According to the nutrition formula management system of claim 13, the nutrition formula management system of any one of claims 1 to 12 includes nutrition formula subject data deletion means configured to, after counting-up to frequency distributions described in the claims is complete, delete data on the nutrition formula subjects managed by the nutrition formula subject management means.

The nutrition formula management system of claim 1 includes the symptom/finding term management means and thus manages the terms for representing the symptom or findings of the nutrition formula subjects.

The nutrition formula management system of claim 1 includes the formulated nutrient term management means and thus manages nutrients to be formulated, as terms.

The nutrition formula management system of claim 1 includes the nutrition formula subject management means and thus represents a list of the symptoms or findings of each of the nutrition formula subjects and a list of formulated nutrients using the terms managed by the symptom/finding term management means and the formulated nutrient term management means and records whether the formulated nutrients have been effective against each of the symptoms or findings of each of the nutrition formula subjects.

The nutrition formula management system of claim 1 includes the symptom/finding-specific effective nutrient list management means and thus counts up, as effective subject frequencies, the frequencies with which each of nutrient lists have been effectively formulated against each of the symptoms or findings of each of the nutrition formula subjects.

The nutrition formula management system of claim 1 includes the symptom/finding-specific, effective frequency-based nutrient suggestion means and thus displays, for each of symptoms or findings of a new nutrition formula subject, a list of nutrients that are managed by the effective nutrient list management means and that have been effective against the symptoms or findings along with the effective subject frequencies and suggests nutrients that seem to be effective against the symptoms or findings of the new nutrition formula subject.

The nutrition formula management system of claim 2 includes the symptom/finding-specific, ineffective nutrient list management means and thus counts up, as ineffective subject frequencies, the frequencies with which each of nutrient lists have been ineffectively formulated against each of the symptoms or findings of each of the nutrition formula subjects.

The nutrition formula management system of claim 2 includes the symptom/finding-specific, effective-ineffective frequency ratio-based nutrient suggestion means and thus displays, for each of symptoms or findings of a new nutrition formula subject, a list of nutrients that have been effective against the symptoms or findings, along with ratios between the effective subject frequencies and the ineffective subject frequencies and suggests nutrients that seem to be effective against the symptoms or findings of the new nutrition formula subject.

The nutrition formula management system of claim 3 includes the symptom/finding-specific effective nutrient pair list management means and thus counts up, as effective subject frequencies, the frequencies with which nutrient pair lists have been effectively formulated against each of the symptoms or findings of each of the nutrition formula subjects.

The nutrition formula management system of claim 3 includes the symptom/finding-specific, effective frequency-based nutrient pair suggestion means and thus displays, for each of symptoms or findings of a new nutrition formula subject, nutrient pair lists that have been effective against the symptoms or findings, along with the effective subject frequencies and suggests nutrient pairs that seem to be effective against the symptoms or findings of the new nutrition formula subject.

The nutrition formula management system of claim 4 includes the symptom/finding-specific ineffective nutrient pair list management means and counts up, as ineffective subject frequencies, the frequencies with which nutrient pair lists have been ineffectively formulated against each of the symptoms or findings of each of the nutrition formula subjects.

The nutrition formula management system of claim 4 includes the symptom/finding-specific, effective-ineffective frequency ratio-based nutrient pair suggestion means and thus displays, for each of symptoms or findings of a new nutrition formula subject, nutrient pair lists that have been effective against the symptoms or findings, along with ratios between the effective subject frequencies and the ineffective subject frequencies and suggests nutrient pairs that seem to be effective against the symptoms or findings of the new nutrition formula subject.

The nutrition formula management system of claim 5 includes the nutrient-specific, symptom/finding-specific effective symptom/finding list management means and thus counts up, as effective symptom/finding frequencies, frequencies with which each of nutrient lists have been effective against each of the symptoms or findings of each of the nutrition formula subjects for which each of the nutrient lists have been formulated.

The nutrition formula management system of claim 5 includes the nutrient-specific, effective frequency-based symptom/finding suggestion means and thus displays, with respect to a nutrient considered to be formulated for a new nutrition formula subject, a list of symptoms and findings that are managed by the nutrient-specific, symptom/finding-specific effective symptom/finding list management means and against which the nutrient has been effective, along with the effective subject frequencies and suggests appropriateness of formula of the nutrient against a symptom or finding of the new nutrition formula subject.

The nutrition formula management system of claim 6 includes the nutrient-specific, symptom/finding-specific ineffective symptom/finding list management means and thus counts up, as ineffective symptom/finding frequencies, frequencies with which each of nutrient lists have been ineffective against each of the symptoms or findings of each of the nutrition formula subjects for which each of the nutrient lists have been formulated.

The nutrition formula management system of claim 6 includes the nutrient-specific, effective-ineffective frequency ratio-based symptom/finding suggestion means and thus displays, with respect to a nutrient considered to be formulated for a new nutrition formula subject, a list of symptoms and findings against which the nutrient has been effective, along with ratios between the effective subject frequencies and the ineffective subject frequencies and suggests appropriateness of formula of the nutrient against a symptom or finding of the new nutrition formula subject.

The nutrition formula management system of claim 7 includes the nutrient-specific, symptom/finding-specific effective symptom/finding list management means and thus counts up, as effective symptom/finding frequencies, frequencies with which each of nutrient lists have been effective against each of the symptoms or findings of each of the nutrition formula subjects for which each of the nutrient lists have been formulated.

The nutrition formula management system of claim 7 includes the nutrient-specific, effective frequency-based symptom/finding suggestion means and thus displays, with respect to a nutrient considered to be formulated for a new nutrition formula subject, a list of symptoms and findings that are managed by the nutrient-specific, symptom/finding-specific effective symptom/finding list management means and against which the nutrient has been effective, along with the effective subject frequencies and to suggest appropriateness of formula of the nutrient against a symptom or finding of the new nutrition formula subject.

The nutrition formula management system of claim 8 includes the nutrient pair-specific, symptom/finding-specific ineffective symptom/finding list management means and thus counts up, as ineffective symptom/finding frequencies, frequencies with which nutrient pair lists have been ineffective against each of the symptoms or findings of each of the nutrition formula subjects for which the nutrient pair lists have been formulated.

The nutrition formula management system of claim 8 includes the nutrient pair-specific, effective-ineffective frequency ratio-based symptom/finding suggestion means and thus displays, with respect to a nutrient pair considered to be formulated for a new nutrition formula subject, a list of symptoms and findings against which the nutrient pair has been effective, along with ratios between the effective subject frequencies and the ineffective subject frequencies and to suggest appropriateness of formula of the nutrient pair against a symptom or a finding of the new nutrition formula subject.

The nutrition formula management system of claim 9 includes the side-effect symptom/finding management means and thus, when a side effect is caused by a formulated nutrient in the nutrition formula subject management means, records a symptom or a finding of the side effect.

The nutrition formula management system of claim 9 includes the nutrient-specific, side-effect symptom/finding list management means and thus counts up frequencies with which each of nutrient lists have caused symptoms or findings as side effects to the nutrition formula subjects for which each of the nutrient lists have been formulated.

The nutrition formula management system of claim 9 includes the nutrient-specific, side-effect frequency-based symptom/finding suggestion means and thus, with respect to a nutrition formula subject who has had a side effect, displays a list of symptoms or findings that are managed by the nutrient-specific, side-effect symptom/finding list management means and that have been previously observed with respect to the nutrient, along with a nutrition formula subject frequency with which the side effect has occurred and suggests a nutrient that may have caused the side effect to the nutrition formula subject.

The nutrition formula management system of claim 10 includes the nutrition pair-specific side-effect symptom/finding list management means, thus includes the side-effect symptom/finding management means configured to, when a formulated nutrient causes a side effect in the nutrition formula subject management means, record a symptom or a finding of the side effect, and counts up the frequencies with which nutrient pair lists have caused symptoms or findings as side effects to the nutrition formula subjects for which the nutrient pair lists have been formulated.

The nutrition formula management system of claim 10 includes the nutrient pair-specific, side-effect frequency-based symptom/finding suggestion means and thus, with respect to a nutrition formula subject who has had a side effect, displays a list of symptoms or findings that are managed by the nutrient pair-specific, side-effect symptom/finding list management means and that have been previously observed with respect to the nutrient pair, along with the nutrition formula subject frequency with which the side effect has occurred and suggests a nutrient pair that may have caused the side effect to the nutrition formula subject.

The nutrition formula management system of claim 11 includes the side-effect symptom/finding-specific nutrient list management means and thus counts up the frequencies with which symptoms or findings as side effects in the side-effect symptom/finding management means have been caused by each of nutrient lists formulated for the nutrition formula subjects.

The nutrition formula management system of claim 11 includes the side-effect symptom/finding frequency-based nutrient suggestion means and thus, with respect to a nutrition formula subject who has had a side effect, displays a list of nutrients that are managed by the side-effect symptom/finding-specific nutrient list management means and that have been formulated against a symptom or a finding of the side effect, along with a nutrition formula subject frequency with which the side effect has occurred and to suggest a nutrient that may have caused the side effect to the nutrition formula subject.

The nutrition formula management system of claim 12 includes the side-effect symptom/finding-specific nutrient pair list management means and thus counts up the frequencies with which symptoms or findings as side effects in the side-effect symptom/finding management means have been caused by nutrient pair lists formulated for the nutrition formula subjects.

The nutrition formula management system of claim 12 includes the side-effect symptom/finding frequency-based nutrient pair suggestion means and thus, with respect to a nutrition formula subject who has had a side effect, display nutrient pair lists that are managed by the side-effect symptom/finding-specific nutrient pair list management means and that have been formulated against a symptom or a finding of the side effect, along with a nutrition formula subject frequency with which the side effect has occurred and to suggest a nutrient pair that may have caused the side effect to the nutrition formula subject.

The nutrition formula management system of claim 13 includes the nutrition formula subject data deletion means and thus, after counting-up to frequency distributions described in the claims is complete, deletes data on the nutrition formula subjects managed by the nutrition formula subject management means.

### BRIEF DESCRIPTION OF THE DRAWINGS

FIG. 1 is a diagram showing symptom/finding term management means, formulated nutrient term management means, and nutrition formula subject management means serving as basic means of a nutrition formula management system of the present application.

FIG. 2 is a diagram showing symptom/finding-specific, effective frequency-based nutrient suggestion means that obtains, as the nutrient-specific effective subject frequencies, the frequencies with which nutrients have been effectively formulated for nutrition formula subjects having a specific symptom or finding (“unable to get up in the morning” in this diagram) in the symptom/finding term management means.

FIG. 3 is a diagram showing symptom/finding-specific, ineffective nutrient list management means that obtains, as the nutrient-specific effective subject frequencies, the frequencies with which nutrients have been ineffectively formulated for nutrition formula subjects having a specific symptom or finding (“unable to get up in the morning” in this diagram) in the symptom/finding term management means.

FIG. 4 is a diagram showing symptom/finding-specific, effective-ineffective frequency ratio-based nutrient suggestion means that obtains the ratios between the nutrient-specific effective subject frequencies, which are the frequencies with which nutrients have been effectively formulated for nutrition formula subjects having a specific symptom or finding (“unable to get up in the morning” in this diagram) in the symptom/finding term management means, and the nutrient-specific ineffective subject frequencies (effective-ineffective ratios).

FIG. 5 is a diagram showing symptom/finding-specific, effective frequency-based nutrient pair suggestion means that obtains, as the nutrient pair-specific effective subject frequencies, the frequencies with which nutrient pairs have been effectively formulated for nutrition formula subjects having a specific symptom or finding (“unable to get up in the morning” in this diagram) in the symptom/finding term management means.

FIG. 6 is a diagram showing symptom/finding-specific, effective-ineffective frequency ratio-based nutrient pair suggestion means that obtains the ratios between the nutrient pair-specific effective subject frequencies, which are the frequencies with which nutrient pairs have been effectively formulated for nutrition formula subjects having a specific symptom or finding (“unable to get up in the morning” in this diagram) in the symptom/finding term management means, and the nutrient pair-specific ineffective subject frequencies (effective-ineffective ratios).

FIG. 7 is a diagram showing that there are obtained the ratios between the effective nutrition subject frequencies, which are the frequencies with which a specific nutrient (“B vitamins” in this diagram) in the formulated nutrient term management means has been effective against each of symptoms or findings of nutrition formula subjects for which the specific nutrient has been formulated, and the ineffective subject frequencies (effective-ineffective ratios).

FIG. 8 is a diagram showing that there are obtained the ratios between the effective nutrition subject frequencies, which are the frequencies with which a specific nutrient pair (“B vitamins” and “iron” in this diagram) in the formulated nutrient term management means has been effective against each of symptoms or findings of nutrition formula subjects for which the specific nutrient pair has been formulated, and the ineffective subject frequencies (effective-ineffective ratios).

FIG. 9 is a diagram showing side-effect frequency nutrient suggestion means that obtains, as the nutrient-specific subject frequencies, the frequencies with which formulated nutrients have caused a specific symptom or finding as a side effect (“nausea” in this diagram) in the symptom/finding term management means to nutrition formula subjects.

FIG. 10 is a diagram showing side-effect symptom/finding frequency-based nutrient pair suggestion means that obtains, as the nutrient pair-specific subject frequencies, the frequencies with which formulated nutrient pairs have caused a specific symptom or finding as a side effect (“nausea” in this diagram) in the symptom/finding term management means to nutrition formula subjects.

### DETAILED DESCRIPTION

A nutrition formula management system includes the server apparatus, the database and the terminal. The server apparatus, the database and the terminal connected through network. The server apparatus is a prior computer. The server apparatus includes: an arithmetic apparatus including the processor, a main storage apparatus, an auxiliary storage apparatus, input apparatus, output apparatus, and communication apparatus. The arithmetic apparatus, the main storage apparatus, the auxiliary storage apparatus, input apparatus, output apparatus, the communication apparatus connected through a bus interface. The arithmetic apparatus includes the processor that can execute an instruction set. The main storage apparatus includes a volatile memory such as a random access memory (RAM). The auxiliary storage apparatus includes a recording medium such as a nonvolatile memory, and a recording method thereof is not limited. The recording medium indicates a hard disk drive (HDD) or a solid state drive (SSD), for example. The input apparatus is, for example, a keyboard device. The output apparatus includes, for example, display as a liquid crystal panel. The communication apparatus is a network interface that can connect to network. The processor of the server apparatus executes the function of the units of the nutrition formula management system including: symptom/finding term management means, formulated nutrient term management means, nutrition formula subject management means, symptom/finding-specific effective nutrient list management means, symptom/finding-specific, effective frequency-based nutrient suggestion means, symptom/finding-specific, ineffective nutrient list management means, symptom/finding-specific, effective-ineffective frequency ratio-based nutrient suggestion means, symptom/finding-specific effective nutrient pair list management means, symptom/finding-specific, effective frequency-based nutrient pair suggestion means, symptom/finding-specific ineffective nutrient pair list management means, symptom/finding-specific, effective-ineffective frequency ratio-based nutrient pair suggestion means, nutrient-specific, symptom/finding-specific effective symptom/finding list management means, nutrient-specific, effective frequency-based symptom/finding suggestion means, nutrient-specific, symptom/finding-specific ineffective symptom/finding list management means, nutrient-specific, effective-ineffective frequency ratio-based symptom/finding suggestion means, nutrient pair-specific, symptom/finding-specific effective symptom/finding list management means, nutrient pair-specific, effective frequency-based symptom/finding suggestion means, nutrient pair-specific, symptom/finding-specific ineffective symptom/finding list management means, nutrient pair-specific, effective-ineffective frequency ratio-based symptom/finding suggestion means, side-effect symptom/finding management means, nutrient-specific, side-effect symptom/finding list management means, nutrient-specific, side-effect frequency-based symptom/finding suggestion means, side-effect symptom/finding management means, nutrient-specific, side-effect symptom/finding list management means, nutrient pair-specific, side-effect frequency-based symptom/finding suggestion means, side-effect symptom/finding-specific nutrient list management means, side-effect symptom/finding frequency-based nutrient suggestion means, side-effect symptom/finding-specific nutrient pair list management means, side-effect symptom/finding frequency-based nutrient pair suggestion means, nutrition formula subject data deletion means or the like. The database is composed of the auxiliary storage apparatus of the server apparatus or the auxiliary storage apparatus independent from the server apparatus. The database stores information managed by the nutrition formula management system. The terminal is a prior computer including a processor.

The present application is a system that runs on a computer.

This computer includes an input device (mouse, keyboard, etc.), an output device (monitor, printer, etc.), a storage device (memory, hard disk), a computing device (CPU), a controller (CPU), and the like, as well as includes a program for performing the means (functions) of the present application.

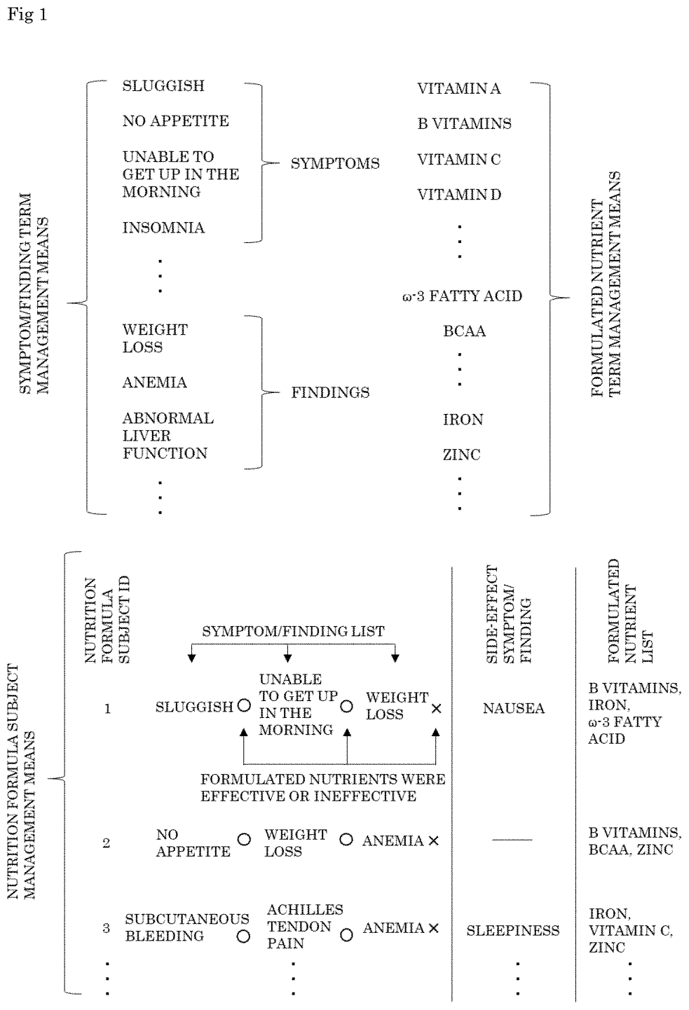

FIG. 1 is a diagram showing symptom/finding term management means, formulated nutrient term management means, and nutrition formula subject management means serving as basic means of a nutrition formula management system of the present application.

The symptom/finding term management means manages a list of (subjective) symptoms such as “sluggish” and “no appetite” and a list of (objective) findings such as “weight loss” and “anemia.” The formulated nutrient term management means manages a list of nutrients used in nutrition formula, such as “vitamin A.”

The nutrition formula subject management means manages a list of nutrition formula subjects to whom nutrients have been formulated, along nutrition formula subject IDs, a list of symptoms or findings of the nutrition formula subjects, a list of the formulated nutrients, whether the listed formulated nutrients have been effective against the listed symptoms or findings of the nutrition formula subjects, and symptoms or findings that have occurred as side effects.

In FIG. 1, ∘ represents effective, and × represents ineffective.

The symptoms and findings are terms managed by the symptom/finding term management means, and the formulated nutrients are terms managed by the formulated nutrient term management means.

These terms may be used as they are, or may be linked via URLs or the like. Also, a term master file in which the terms are managed using ID Nos, may be previously created, and the terms may be described using the ID Nos.

Each management means may be implemented using any means, such as an object RDB described in an XML or JSON format.

Although not shown, it is also useful to additionally manage information, such as the past disease names or oral medicine names in use of the nutrition formula subjects. It is also useful to link the nutrition formula subjects to records, such as electronic health records.

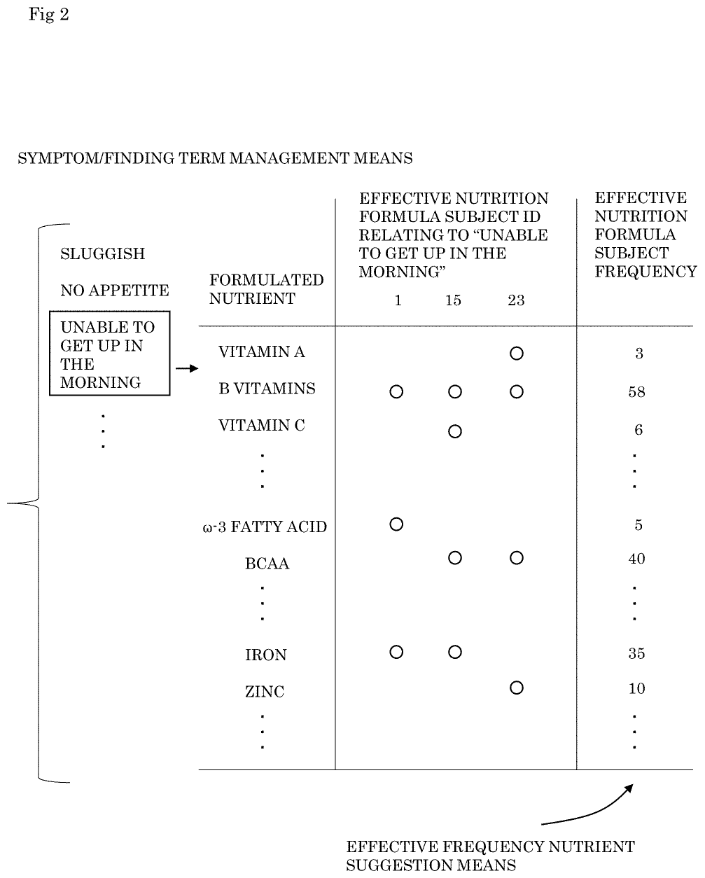

FIG. 2 is a diagram showing symptom/finding-specific, effective frequency-based nutrient suggestion means that obtains, as the nutrient-specific effective subject frequencies, the frequencies with which nutrients have been effectively formulated for nutrition formula subjects having a specific symptom or finding (“unable to get up in the morning” in this diagram) in the symptom/finding term management means.

The symptom/finding-specific, effective frequency-based nutrient suggestion means lists nutrition formula subjects who have the specific symptom “unable to get up in the morning” and against the specific symptom of whom nutrients have been effectively formulated among those managed by the nutrition formula subject management means and obtains the effective nutrition formula subject frequencies by counting up nutrients in nutrient lists formulated for the nutrition formula subjects.

While the effective nutrition formula subject frequency is increased with respect to nutrients that have been really effective among the formulated nutrients, the frequency remains low with respect to nutrients that have not been effective but have been formulated occasionally. Use of the nutrient-specific effective nutrition formula subject frequencies allows for suggesting a nutrient to be formulated against the specific symptom or finding (“unable to get up in the morning” in this diagram) (symptom/finding-specific, effective frequency-based nutrient suggestion means).

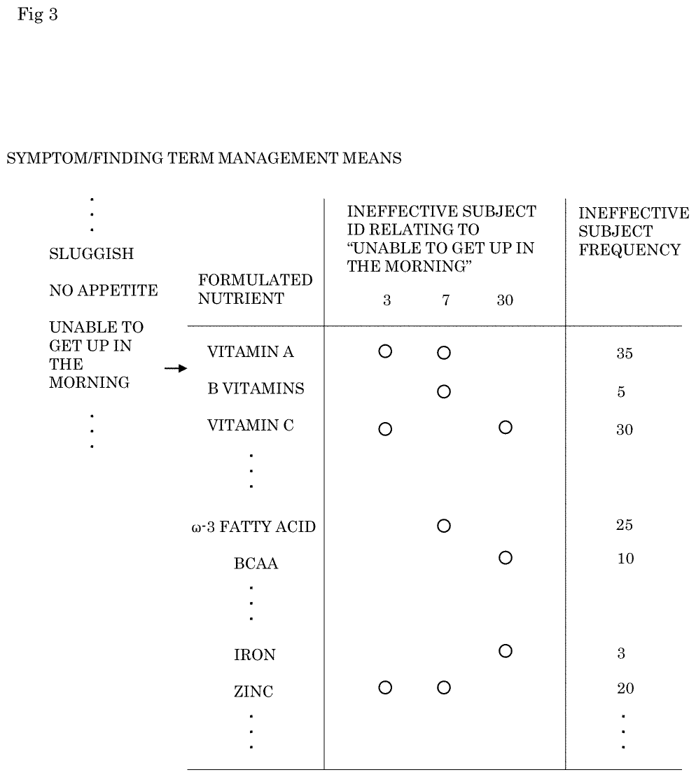

FIG. 3 is a diagram showing symptom/finding-specific, ineffective nutrient list management means that obtains, as the nutrient-specific effective subject frequencies, the frequencies with which nutrients have been ineffectively formulated for nutrition formula subjects having a specific symptom or finding (“unable to get up in the morning” in this diagram) in the symptom/finding term management means.

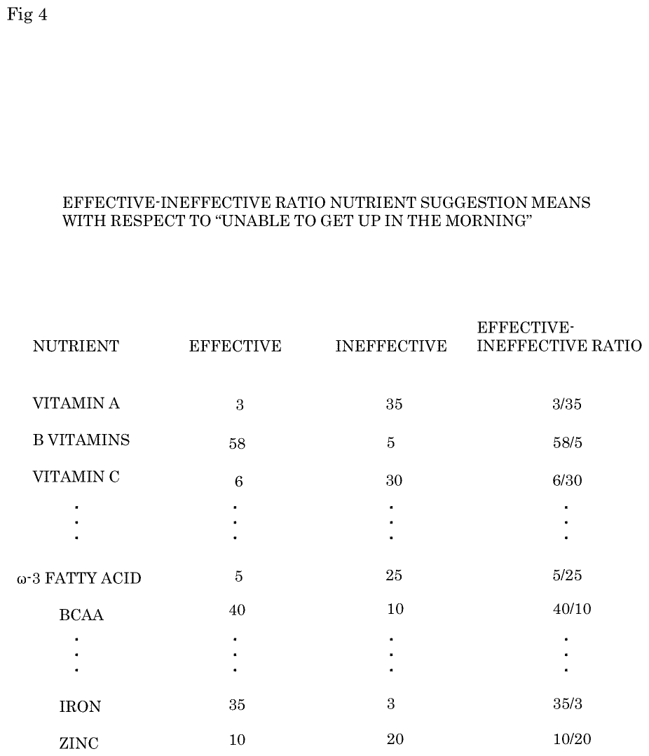

FIG. 4 is a diagram showing symptom/finding-specific, effective-ineffective frequency ratio-based nutrient suggestion means that obtains the ratios between the nutrient-specific effective subject frequencies, which are the frequencies with which nutrients have been effectively formulated for nutrition formula subjects having a specific symptom or finding (“unable to get up in the morning” in this diagram) in the symptom/finding term management means, and the nutrient-specific ineffective subject frequencies (effective-ineffective ratios).

While the effective nutrition formula subject frequency is increased with respect to nutrients that have been really effective among the formulated nutrients, the frequency remains low with respect to nutrients that have not been effective but have been formulated occasionally. However, the frequency of a nutrient that has been formulated more often although the nutrient has not been really effective may be increased, and There is a risk of giving false suggestions.

This risk can be avoided by obtaining the ratios between the nutrient-specific effective subject frequencies, which are the frequencies with which nutrients have been effectively formulated for nutrition formula subjects, and the nutrient-specific ineffective subject frequencies (effective-ineffective ratio). (symptom/finding-specific, effective-ineffective frequency ratio-based nutrient suggestion means).

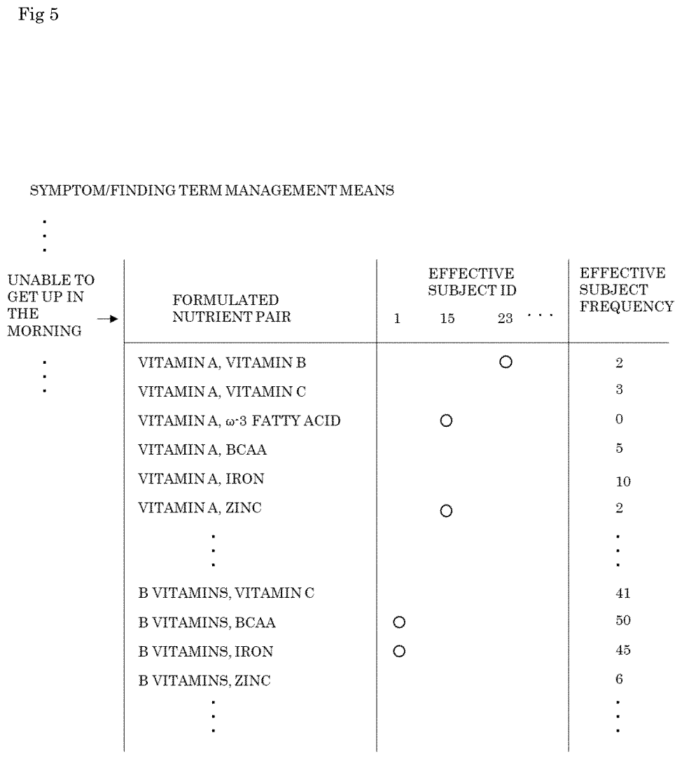

FIG. 5 is a diagram showing symptom/finding-specific, effective frequency-based nutrient pair suggestion means that obtains, as the nutrient pair-specific effective subject frequencies, the frequencies with which nutrient pairs have been effectively formulated for nutrition formula subjects having a specific symptom or finding (“unable to get up in the morning” in this diagram) in the symptom/finding term management means.

When multiple nutrients are administered simultaneously, the effects thereof may be increased (synergy effects) or may conversely cancel each other and be reduced (cancel effects). By measuring the nutrition pair-specific effective subject frequencies, an effective nutrition pair can be suggested (symptom/finding-specific, effective frequency-based nutrition pair suggestion means).

If the effective nutrition formula subject frequency of a nutrition pair is higher than that of a single nutrient, this means that synergy effects have been produced; if the frequency is conversely lower, this means that cancel effects have been produced.

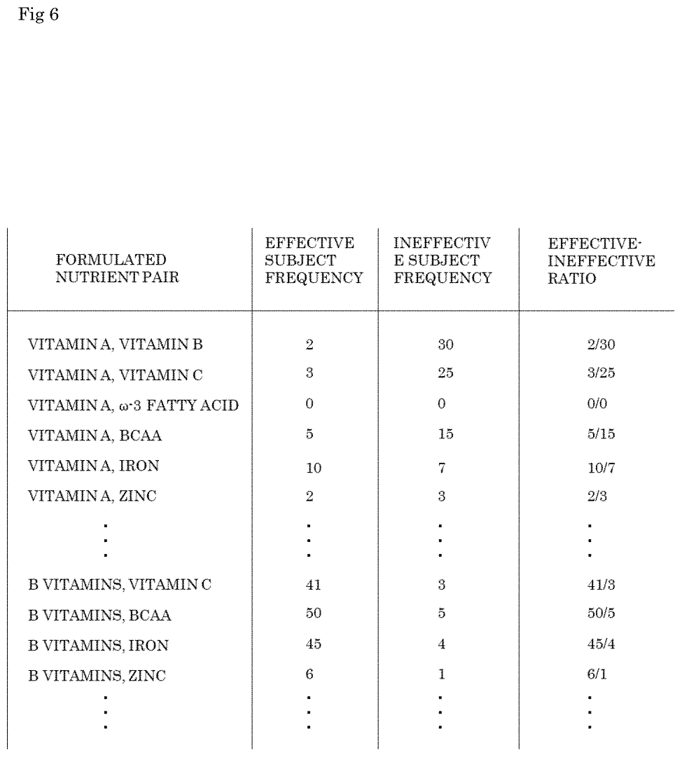

FIG. 6 is a diagram showing symptom/finding-specific, effective-ineffective frequency ratio-based nutrient pair suggestion means that obtains the ratios between the nutrient pair-specific effective subject frequencies, which are the frequencies with which nutrient pairs have been effectively formulated for nutrition formula subjects having a specific symptom or finding (“unable to get up in the morning” in this diagram) in the symptom/finding term management means, and the nutrient pair-specific ineffective subject frequencies (effective-ineffective ratios).

While the effective nutrition formula subject frequency is increased with respect to nutrient pairs that have been really effective among the formulated nutrition pairs, the frequency remains low with respect to nutrition pairs that have not been effective but have been formulated occasionally.

However, the frequency of a nutrient pair that has been formulated more often although the nutrient has not been really effective may be increased, and this nutrient pair may be erroneously suggested.

This risk can be avoided by obtaining the ratios between the nutrient pair-specific effective subject frequencies, which are the frequencies with which nutrient pairs have been effectively formulated for nutrition formula subjects, and the nutrient pair-specific ineffective subject frequencies (effective-ineffective ratios). (symptom/finding-specific, effective-ineffective frequency ratio-based nutrient pair suggestion means).

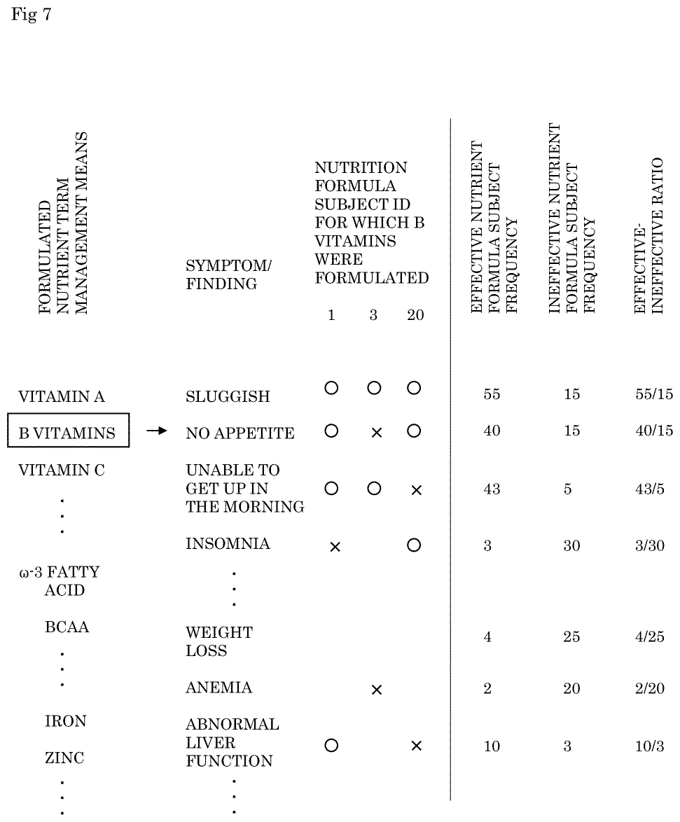

In FIG. 7, there are obtained the ratios between the effective nutrition subject frequencies, which are the frequencies with which a specific nutrient (“B vitamins” in this diagram) in the formulated nutrient term management means has been effective against each of symptoms or findings of nutrition formula subjects for which the specific nutrient has been formulated, and the ineffective subject frequencies (nutrient-specific, symptom/finding-specific effective symptom/finding list management means) (nutrient-specific, effective frequency-based symptom/finding suggestion means).

The ineffective nutrition formula subject frequencies are also obtained. There are also obtained the ratios between the nutrition formula subject frequencies, which are the frequencies with which the specific nutrient has been effective for each of symptoms and findings of the nutrition formula subjects for which the specific nutrient has been formulated, and the nutrition formula subject frequencies, which are the frequencies with which the specific nutrient has been ineffective (nutrient-specific, effective-ineffective frequency ratio-based symptom/finding suggestion means).

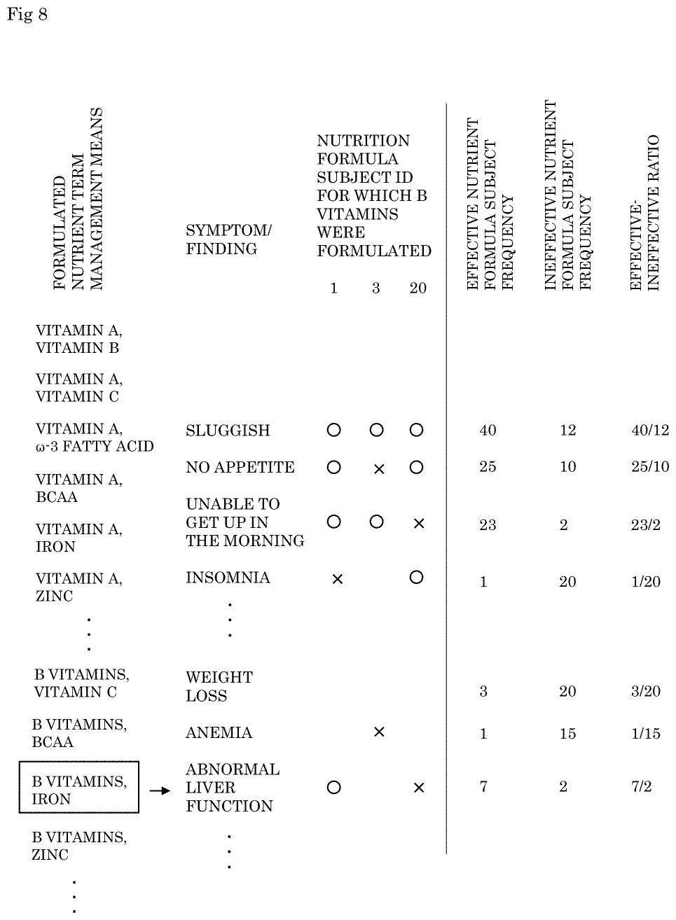

In FIG. 8, there are obtained the effective nutrition subject frequencies, which are the frequencies with which a specific nutrient pair (“B vitamins” and “iron” in this diagram) in the formulated nutrient term management means has been effective against each of symptoms or findings of nutrition formula subjects for which the specific nutrient pair has been formulated (nutrient pair-specific, symptom/finding-specific, effective symptom/finding list management means) (nutrient pair-specific, effective frequency-based symptom/finding suggestion means).

The ineffective nutrition formula subject frequencies are also obtained. There are also obtained the ratios between the nutrition formula subject frequencies, which are the frequencies with which the specific nutrient has been effective for each of symptoms and findings of the nutrition formula subjects for which the specific nutrient has been formulated and the nutrition formula subject frequencies, which are the frequencies with which the specific nutrient has been ineffective (nutrient pair-specific, effective-ineffective frequency ratio-based symptom/finding suggestion means).

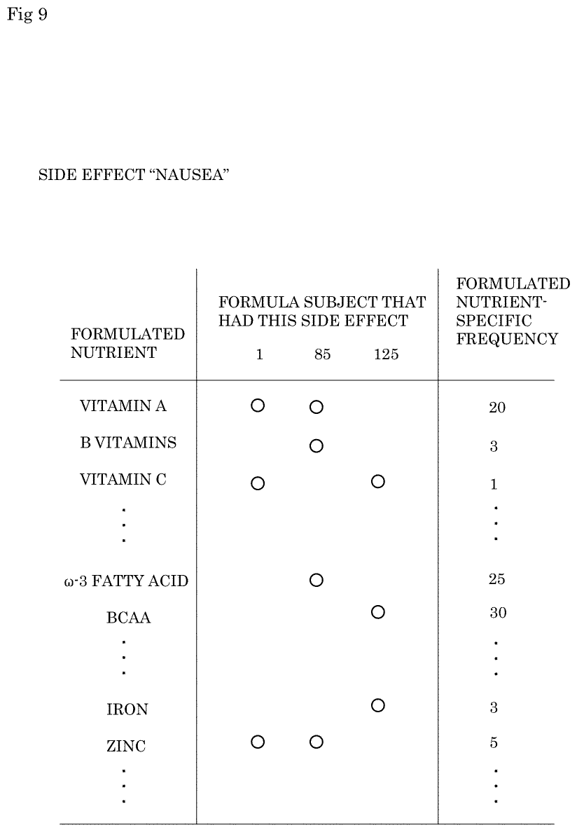

FIG. 9 is a diagram showing side-effect frequency nutrient suggestion means that obtains, as the nutrient-specific subject frequencies, the frequencies with which formulated nutrients have caused a specific symptom or finding as a side effect (“nausea” in this diagram) in the symptom/finding term management means to nutrition formula subjects.

The side-effect frequency nutrient suggestion means lists nutrition formula subjects who have had the specific side effect “nausea” due to the formulation of nutrients for them and obtains the formulated nutrient-specific nutrition formula subject frequencies by counting up nutrients in nutrient lists formulated for the nutrition formula subjects.

Although both are “nausea,” “nausea” that is a symptom or finding to be improved by formulating nutrition and “nausea” that is an unexpected symptom or finding caused by formulation of nutrition are quite different. For this reason, nutrients lists formulated for these types of nausea are managed in the symptom/finding term management means separately.

While the nutrition formula subject frequency, with which the side effect has occurred, is increased with respect to nutrients that have really caused the side effect among the formulated nutrients, the frequency remains low with respect to nutrients that have not caused the side effect but have been formulated occasionally.

Use of the nutrient-specific nutrition formula subject frequencies, with which the side effect has occurred, allows for suggesting a nutrient that has caused the specific side effect (“nausea” in this diagram) (side-effect symptom/finding frequency-based nutrient suggestion means).

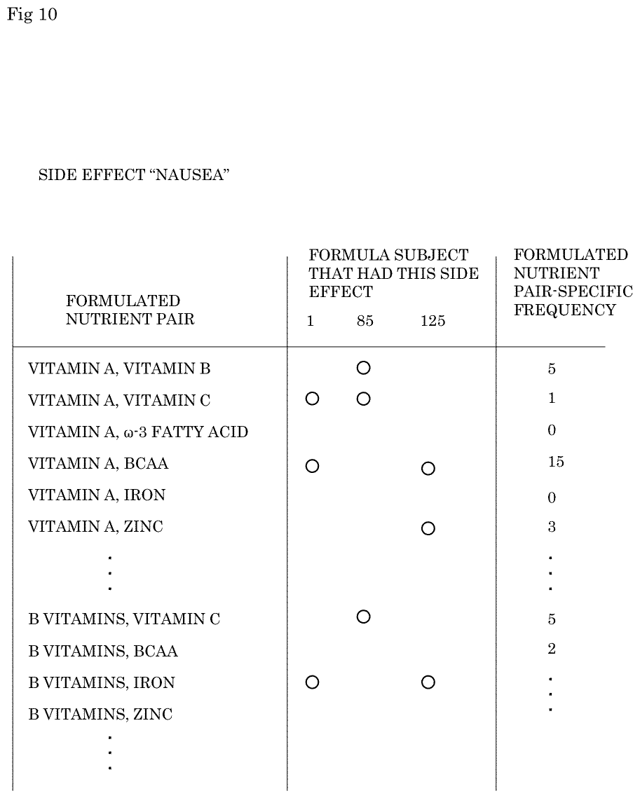

FIG. 10 is a diagram showing side-effect symptom/finding frequency-based nutrient pair suggestion means that obtains, as the nutrient pair-specific subject frequencies, the frequencies with which formulated nutrient pairs have caused a specific symptom or finding as a side effect (“nausea” in this diagram) in the symptom/finding term management means to nutrition formula subjects.

The side-effect symptom/finding frequency-based nutrient pair suggestion means lists nutrition formula subjects who have had the specific side effect “nausea” due to the formula of nutrients for them and obtains the formulated nutrient pair-specific nutrition formula subject frequencies by counting up nutrient pairs in nutrient lists formulated for the nutrition formula subjects.

While the nutrition formula subject frequency, with which the side effect has occurred, is increased with respect to nutrient pairs that have really caused the side effect among the formulated nutrient pairs, the frequency remains low with respect to nutrient pairs that have not caused the side effect but have been formulated occasionally.

Use of the nutrient pair-specific nutrition formula subject frequencies, with which the side effect has occurred, allows for suggesting a nutrition pair that has caused the specific side effect (“nausea” in this diagram) (side-effect frequency-based nutrient pair suggestion means).

When multiple nutrients are administered simultaneously, the effects thereof may be increased (synergy effects) or may conversely cancel each other and be reduced (cancel effects).

By measuring the nutrition pair-specific frequencies of the subjects who have had the side effect, an effective nutrition pair or a nutrition pair that should be avoided can be suggested.

The nutrition formula subject management means is recording a list of symptoms and findings of the nutrition formula subjects, a list of formulated nutrients, whether the nutrients have been effective or ineffective against the symptoms and findings, and symptoms and findings that are side effects.

While it is useful to keep holding these records, for example, when additional analysis is needed, the cost for managing the records tends to increase.

What is directly required when formulating nutrition is the frequencies with which combinations of formulated nutrients and symptoms or findings have been effective or ineffective, and the records of the individual nutrition formula subjects used to calculate the frequencies are not required.

For this reason, the records of the individual nutrition formula subjects used to calculate the frequencies with which combinations of formulated nutrients and symptoms or findings have been effective or ineffective are deleted, and only the resulting frequency distributions are used. Thus, the object of assisting in formulating nutrition with a small amount of data can be accomplished.

By distributing only the frequency distributions to multiple nutrition formulators, nutrition can be efficiently formulated at many facilities.

When deleting the records of the individual nutrition formula subjects, the records may be deleted shortly after the frequency is calculated in each case, or the records may be deleted all at once after the frequency calculation is complete.

Since many matters remain unknown with respect to the relationships between nutrients and symptoms or findings, it is not necessarily useful to deductively consider nutrition formula as is done in Patent Literature 3.

It seems to be useful to inductively assist in formulating nutrition by obtaining the frequency distribution between both.

While an embodiment has been described, the nutrients, symptoms or findings, and frequencies shown in the diagrams are only illustrative. In practice, these are, of course, defined and measured more precisely.

The specific configuration of the present invention is not limited to the embodiment, and design changes or the like are also included in the present invention without departing from the spirit and scope of the present invention.

For example, while nutrient formula and the symptoms or findings and side effects to be improved have been described in the present invention, the same configuration can be used to provide assistance information that relates to medicines, symptoms or findings of diseases to be treated, and the side effects of the medicines and that is useful to prescribe medicines.

1. A nutrition formula management system comprising:
(i) symptom/finding term management means configured to manage terms for representing symptoms or findings of nutrition formula subjects;
(ii) formulated nutrient term management means configured to manage nutrients to be formulated, as terms;
(iii) nutrition formula subject management means configured to represent a list of the symptoms or findings of each of the nutrition formula subjects and a list of formulated nutrients using the terms managed by the symptom/finding term management means and the formulated nutrient term management means and to record whether the formulated nutrients have been effective against each of the symptoms or findings of each of the nutrition formula subjects;
(iv) symptom/finding-specific effective nutrient list management means configured to count up, as effective subject frequencies, frequencies with which each of nutrient lists have been effectively formulated against each of the symptoms or findings of each of the nutrition formula subjects; and
symptom/finding-specific, effective frequency-based nutrient suggestion means configured to display, with respect to each of symptoms or findings of a new nutrition formula subject, a list of nutrients that are managed by the effective nutrient list management means and that have been effective against the symptoms or findings, along with the effective subject frequencies and to suggest nutrients that seem to be effective against the symptoms or findings of the new nutrition formula subject.

2. The nutrition formula management system of claim 1, comprising:
symptom/finding-specific, ineffective nutrient list management means configured to count up, as ineffective subject frequencies, frequencies with which each of nutrient lists have been ineffectively formulated against each of the symptoms or findings of each of the nutrition formula subjects; and
symptom/finding-specific, effective-ineffective frequency ratio-based nutrient suggestion means configured to display, with respect to each of symptoms or findings of a new nutrition formula subject, a list of nutrients that have been effective against the symptoms or findings, along with ratios between the effective subject frequencies and the ineffective subject frequencies and to suggest nutrients that seem to be effective against the symptoms or findings of the new nutrition formula subject.

3. The nutrition formula management system of claim 1, comprising:
symptom/finding-specific effective nutrient pair list management means configured to count up, as effective subject frequencies, frequencies with which nutrient pair lists have been effectively formulated against each of the symptoms or findings of each of the nutrition formula subjects; and
symptom/finding-specific, effective frequency-based nutrient pair suggestion means configured to display, with respect to each of symptoms or findings of a new nutrition formula subject, nutrient pair lists that have been effective against the symptoms or findings, along with the effective subject frequencies and to suggest nutrient pairs that seem to be effective against the symptoms or findings of the new nutrition formula subject.

4. The nutrition formula management system of claim 1, comprising:
symptom/finding-specific ineffective nutrient pair list management means configured to count up, as ineffective subject frequencies, frequencies with which nutrient pair lists have been ineffectively formulated against each of the symptoms or findings of each of the nutrition formula subjects; and
symptom/finding-specific, effective-ineffective frequency ratio-based nutrient pair suggestion means configured to display, with respect to each of symptoms or findings of a new nutrition formula subject, nutrient pair lists that have been effective against the symptoms or findings, along with ratios between the effective subject frequencies and the ineffective subject frequencies and to suggest nutrient pairs that seem to be effective against the symptoms or findings of the new nutrition formula subject.

5. The nutrition formula management system of claim 1, comprising:
nutrient-specific, symptom/finding-specific effective symptom/finding list management means configured to count up, as effective symptom/finding frequencies, frequencies with which each of nutrient lists have been effective against each of the symptoms or findings of each of the nutrition formula subjects for which each of the nutrient lists have been formulated; and
nutrient-specific, effective frequency-based symptom/finding suggestion means configured to display, with respect to a nutrient considered to be formulated for a new nutrition formula subject, a list of symptoms and findings that are managed by the nutrient-specific, symptom/finding-specific effective symptom/finding list management means and against which the nutrient has been effective, along with the effective subject frequencies and to suggest appropriateness of formula of the nutrient against a symptom or finding of the new nutrition formula subject.

6. The nutrition formula management system of claim 1, comprising:
nutrient-specific, symptom/finding-specific ineffective symptom/finding list management means configured to count up, as ineffective symptom/finding frequencies, frequencies with which each of nutrient lists have been ineffective against each of the symptoms or findings of each of the nutrition formula subjects for which each of the nutrient lists have been formulated; and
nutrient-specific, effective-ineffective frequency ratio-based symptom/finding suggestion means configured to display, with respect to a nutrient considered to be formulated for a new nutrition formula subject, a list of symptoms and findings against which the nutrient has been effective, along with ratios between the effective subject frequencies and the ineffective subject frequencies and to suggest appropriateness of formula of the nutrient against a symptom or finding of the new nutrition formula subject.

7. The nutrition formula management system of claim 1, comprising:
nutrient pair-specific, symptom/finding-specific effective symptom/finding list management means configured to count up, as effective symptom/finding frequencies, frequencies with which nutrient pair lists have been effective against each of the symptoms or findings of each of the nutrition formula subjects for which the nutrient pair lists have been formulated; and nutrient pair-specific, effective frequency-based symptom/finding suggestion means configured to display, with respect to a nutrient pair considered to be formulated for a new nutrition formula subject, a list of symptoms and findings that are managed by the nutrient-specific, symptom/finding-specific effective symptom/finding list management means and against which the nutrient pair has been effective, along with the effective subject frequencies and to suggest appropriateness of formula of the nutrient pair against a symptom or a finding of the new nutrition formula subject.

8. The nutrition formula management system of claim 1, comprising:
nutrient pair-specific, symptom/finding-specific ineffective symptom/finding list management means configured to count up, as ineffective symptom/finding frequencies, frequencies with which nutrient pair lists have been ineffective against each of the symptoms or findings of each of the nutrition formula subjects for which the nutrient pair lists have been formulated; and
nutrient pair-specific, effective-ineffective frequency ratio-based symptom/finding suggestion means configured to display, with respect to a nutrient pair considered to be formulated for a new nutrition formula subject, a list of symptoms and findings against which the nutrient pair has been effective, along with ratios between the effective subject frequencies and the ineffective subject frequencies and to suggest appropriateness of formula of the nutrient pair against a symptom or a finding of the new nutrition formula subject.

9. The nutrition formula management system of claim 1, comprising:
side-effect symptom/finding management means configured to, when a side effect is caused by a formulated nutrient in the nutrition formula subject management means, record a symptom or a finding of the side effect;
nutrient-specific, side-effect symptom/finding list management means configured to count up frequencies with which each of nutrient lists have caused symptoms or findings as side effects to the nutrition formula subjects for which each of the nutrient lists have been formulated; and
nutrient-specific, side-effect frequency-based symptom/finding suggestion means configured to, with respect to a nutrition formula subject who has had a side effect, display a list of symptoms or findings that are managed by the nutrient-specific, side-effect symptom/finding list management means and that have been previously observed with respect to the nutrient, along with a nutrition formula subject frequency with which the side effect has occurred and to suggest a nutrient that may have caused the side effect to the nutrition formula subject.

10. The nutrition formula management system of claim 1, comprising:
side-effect symptom/finding management means configured to, when a side effect is caused by a formulated nutrient in the nutrition formula subject management means, record a symptom or a finding of the side effect;
nutrient-specific, side-effect symptom/finding list management means configured to count up frequencies with which nutrient pair lists have caused symptoms or findings as side effects to the nutrition formula subjects for which the nutrient pair lists have been formulated; and
nutrient pair-specific, side-effect frequency-based symptom/finding suggestion means configured to, with respect to a nutrition formula subject who has had a side effect, display a list of symptoms or findings that are managed by the nutrient pair-specific, side-effect symptom/finding list management means and that have been previously observed with respect to the nutrient pair, along with a nutrition formula subject frequency with which the side effect has occurred and to suggest a nutrient pair that may have caused the side effect to the nutrition formula subject.

11. The nutrition formula management system of claim 9, comprising:
side-effect symptom/finding-specific nutrient list management means configured to count up frequencies with which symptoms or findings as side effects in the side-effect symptom/finding management means have been caused by each of nutrient lists formulated for the nutrition formula subjects; and
side-effect symptom/finding frequency-based nutrient suggestion means configured to, with respect to a nutrition formula subject who has had a side effect, display nutrient lists that are managed by the side-effect symptom/finding-specific nutrient list management means and that have been formulated against a symptom or a finding of the side effect, along with a nutrition formula subject frequency with which the side effect has occurred and to suggest a nutrient that may have caused the side effect to the nutrition formula subject.

12. The nutrition formula management system of claim 9, comprising:
side-effect symptom/finding-specific nutrient pair list management means configured to count up frequencies with which symptoms or findings as side effects in the side-effect symptom/finding management means have been caused by nutrient pair lists formulated for the nutrition formula subjects; and
side-effect symptom/finding frequency-based nutrient pair suggestion means configured to, with respect to a nutrition formula subject who has had a side effect, display nutrient pair lists that are managed by the side-effect symptom/finding-specific nutrient pair list management means and that have been formulated against a symptom or a finding of the side effect, along with a nutrition formula subject frequency with which the side effect has occurred and to suggest a nutrient pair that may have caused the side effect to the nutrition formula subject.

13. The nutrition formula management system of claim 1, comprising nutrition formula subject data deletion means configured to, after counting-up to frequency distributions described in the claims is complete, delete data on the nutrition formula subjects managed by the nutrition formula subject management means.
